(19) United States
(12) Patent Application Publication
(10) Pub. No.: US 2007/0112399 A1
(43) Pub. Date: May 17, 2007

(54) Thermoceramic lifter of thermotherapy apparatus

(76) Inventor: Hee-Jong Baek, Gwangiu (KR)

Correspondence Address: LADAS & PARRY LLP, 224 SOUTH MICHIGAN AVENUE, SUITE 1600, CHICAGO, IL, 60604, US

(21) Appl. No.: 11/589,450

(22) Filed: Oct. 30, 2006

(30) Foreign Application Priority Data
Oct. 31, 2005 (KR) 2005-0103104

(51) Int. Cl. A61N 5/06

(52) U.S. Cl. 607/90

(57) ABSTRACT

A thermoceramic lifter of a thermotherapy apparatus that applies pressure to acupressure points of a user and smoothly and silently raises and lowers itself as needed, applies pressure comfortably to local areas as specified by the user, and has a simple lifting structure, is provided. The lifter (200) of a thermoceramic unit (100) lifts and lowers the thermoceramic unit (100) at a predetermined location along a lengthwise movement of the thermoceramic unit (100). The lengthwise movement is enabled by a conveyor in the thermotherapy apparatus. The lifter (200) includes a box-shaped guiding block (220), a geared motor (210) installed opposite to the open side of the guiding block (220), a sliding block (230) raised and lowered within the guiding block (220) linear movement, and a cover plate (240) to cover the open side of the guiding block (220).

### BACKGROUND OF THE INVENTION

1. Field of the Invention

The present invention relates to a thermoceramic lifter of a thermotherapy apparatus, and more particularly, to a thermoceramic lifter of a thermotherapy apparatus that applies pressure to acupressure points of a user and smoothly and silently raises and lowers itself as needed, applies pressure comfortably to local areas as specified by the user, and has a simple lifting structure for a reduced manufacturing cost and ability to be mass-produced.

2. Description of the Related Art

The human body has many acupressure points. When these points have pressure and massaging applied to them, the local cells exhibit a repelling characteristic, in which heat is radiated and waste materials are expelled therefrom. Treatments which exploit the clearing of acupressure points so that they can be treated have been developed. Modern ailments include back pain and neck and shoulder pain, which may be attributable largely to increased time spent in a seated position and a lack of exercise. The widespread use of computers can be seen as an aggravation to these problems. Thus, physiotherapy devices equipped with helium and infrared lamps, etc. are being used in the home and facilities outside the home. Devices that are now widely used include thermotherapy devices for applying pressure and heat to treat afflicted regions of the body, and devices employing high frequency lamps.

Thermotherapy apparatuses use helium lamps, infrared lamps, and white incandescent bulbs to radiate light, heat, and infrared rays for thermotherapy to activate cells and provide physiotherapeutic treatment. Such a thermotherapy apparatus 300 (shown in FIG. 4) uses a thermoceramic unit 100 that moves across a user's acupressure points along the spinal vertebrae, guided on rails of the thermotherapy apparatus. The thermoceramic unit 100 is also raised and lowered by a lifter 200 to stimulate the spine while providing acupressure and thermotherapy through heat and infrared rays emitted by the thermoceramic unit 100, in order to treat a variety of ailments.

However, in this thermoceramic unit 100 of the thermotherapy apparatus, the lifter 200 emits severe vibration and noise when subjected to load, so that use thereof is unpleasant. Also, because the lifter 200 of the thermoceramic unit 100 is structurally complex, it is subject to frequent malfunctions, reducing product reliability, and also has a high product cost.

### SUMMARY OF THE INVENTION

Accordingly, the present invention is directed to a leg massager using air that substantially obviates one or more problems due to limitations and disadvantages of the related art.

An object of the present invention is to provide a thermoceramic lifter of a thermotherapy apparatus capable of elevating a sliding block disposed inside a square guide block with a geared motor, using a leveling cam, to not only block the emission of noise during elevating, but also to simplify the structure to prevent malfunctioning and enable mass-production.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, there is provided a lifter (200) of a thermoceramic unit (100) for a thermotherapy apparatus, for lifting and lowering the thermoceramic unit (100) at a predetermined location along a lengthwise movement of the thermoceramic unit (100), the lengthwise movement enabled by a conveyor in the thermotherapy apparatus, the lifter (200) including: a box-shaped guiding block (220) open at a side thereof, connected to the conveyor, and installed below the thermoceramic unit (100); a geared motor (210) installed opposite to the open side of the guiding block (220); a sliding block (230) installed in the guiding block (220) within which the sliding block (230) is raised and lowered through a converting driving member that converts a rotating movement of the geared motor (210) to a linear movement; and a cover plate (240) for covering the open side of the guiding block (220).

Here, the sliding block (230) may include: a passage (232) formed therein on surfaces proximal to the guiding block (220) for enabling a smooth elevation thereof; and an elevating hole (234) formed horizontally along a middle of the sliding block (230) for receiving driving force from the geared motor (210) through the converting driving member, wherein the converting driving member includes an eccentric cam (224) installed on a rotating shaft (212) of the geared motor (210) and inserted into the guiding block (220), an eccentric shaft (226) fitted in an eccentric hole (225) formed in an eccentric portion of the eccentric cam (224), and a driving bearing (228) inserted over the eccentric shaft (226) and mounted in the elevating hole (234).

The link may further include a rotating shaft support bearing (222) installed between the eccentric cam (224) and the guiding block (220), for enabling a smooth rotation of the eccentric cam (224).

The lifter may further include: a movement detecting pin (236) protruding from the sliding block (230) toward the cover plate (240); a pin through-hole (246) elongatedly formed in the cover plate (240), the movement detecting pin (236) moving in a vertical direction in the pin through-hole (246); and an upper limit switch (244) and a lower limit switch (242) for detecting a vertical displacement of the movement detecting pin (236).

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a disassembled perspective view of a thermoceramic unit and a lifter according to the first embodiment of the present invention;

FIG. 2 is an exploded perspective view of a lifter according to the first embodiment of the present invention;

FIGS. 3I through 3IV are plan views of a lifter in different rotational positions, according to the first embodiment of the present invention; and

FIG. 4 is a schematic sectional view of a thermotherapy apparatus, according to the related art.

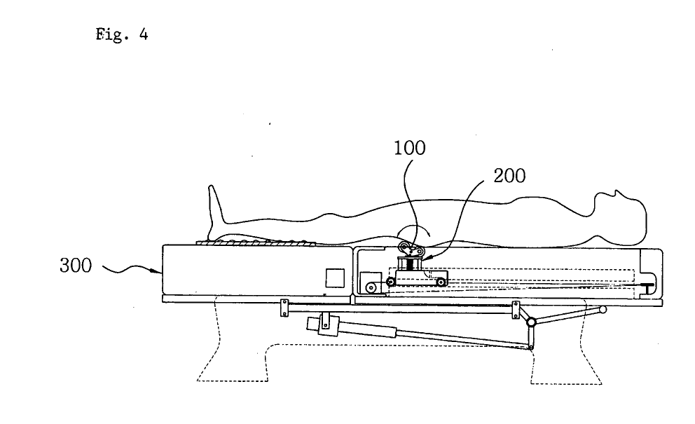

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

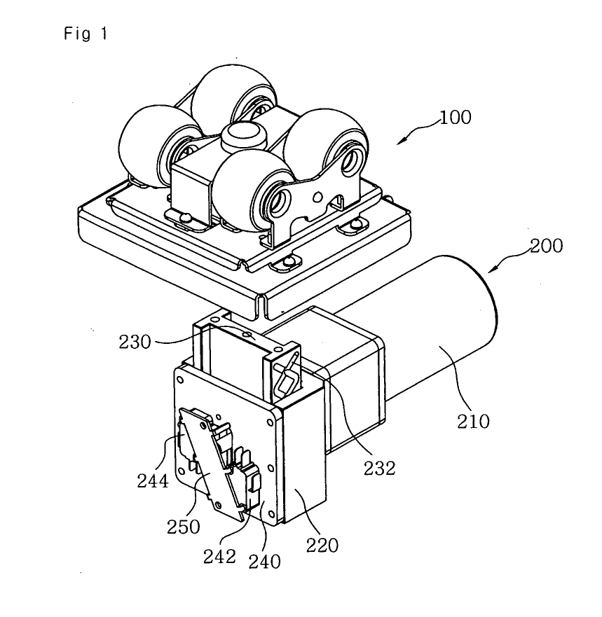

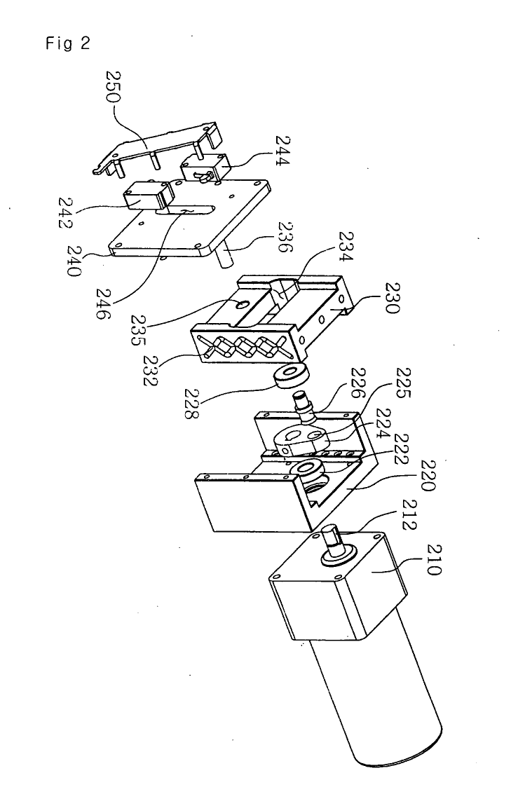

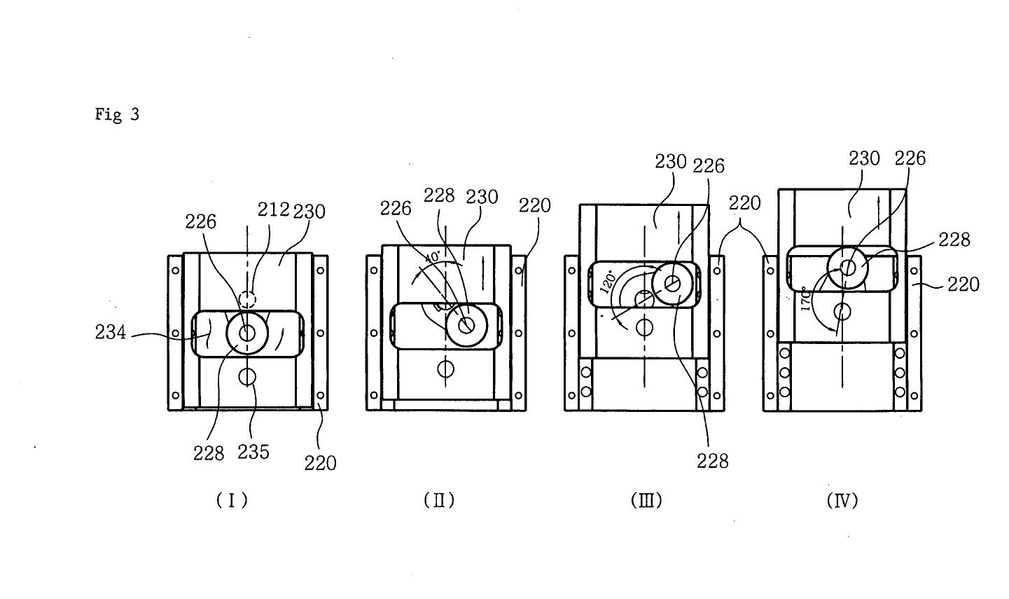

FIG. 1 is a disassembled perspective view of a thermoceramic unit and a lifter according to the first embodiment of the present invention, FIG. 2 is an exploded perspective view of a lifter according to the first embodiment of the present invention, and FIGS. 3I through 3IV are plan views of a lifter in different rotational positions, according to the first embodiment of the present invention.

Referring to FIGS. 1 through 3, the lifter 200 for a thermoceramic unit 100 of a thermotherapy apparatus 300 according to embodiments of the present invention is a device that evenly elevates the thermoceramic unit 100 without emitting excessive noise. This lifter 200 includes a box-shaped guiding block 220 open at a side thereof, a geared motor 210 having a gear mechanism and installed opposite to the open side of the guiding block 220, a sliding block 230 inserted into the guiding block 220 within which it is raised and lowered by the geared motor 210, and a cover plate 240 for covering the open side of the guiding block 220.

Here, the sliding block 230 has a passage 232 formed therein on surfaces proximal to the guiding block 220 so that smooth elevation thereof can be realized. This type of elevating motion is made possible by an elevating hole 234 formed horizontally along the middle of the sliding block 230, which receives driving force from the geared motor 210. In other words, the rotating shaft 212 of the geared motor 210 is inserted and installed into the guiding block 220, on which an eccentric cam 224 is installed. The eccentric portion of the eccentric cam 224 has an eccentric hole formed 225 therein, in which an eccentric shaft 226 is fitted. A driving bearing 228 is inserted over the eccentric shaft 226, and transfers the driving force. A rotating shaft support bearing 222 is disposed between the eccentric cam 224 and the guiding block 220 to allow smooth rotation of the eccentric cam 224.

The sliding block 230 has a pin hole 235 formed below the elevating hole 234. The upward and downward displacement of the sliding block 230 is detected by a movement detecting pin 236 inserted in the pin hole 235. That is, the pin through-hole 246 is formed vertically in the center of the cover plate 240 to allow the movement detecting pin 236 to move, and the displacement of the movement detecting pin 236 that moves up and down within the pin through-hole 246 is detected by a upward limiting switch 244 and a downward limiting switch 242 installed at appropriate locations at the top and bottom of the cover plate 240.

Here, the movement detecting pin 236 moving vertically within the pin through-hole 246 may be protected by a protective plate 250 installed on the cover plate 240. The protective plate 250, the cover plate 240, the guiding block 220, and the geared motor 210 are bolted or otherwise firmly coupled together.

The operation of the above-structured lifter, as shown in FIG. 3I, is as follows. When the eccentric shaft 226 installed in the eccentric hole 225 of the eccentric cam 224 is at the lowest point, the driving bearing 228 installed in the eccentric shaft 226 is disposed at the center of the elevating hole 234 of the sliding block 230. Here, the driving bearing 228 is disposed at the inner upper surface of the elevating hole 234 and is separated from the lower surface.

Next, when the geared motor 210 rotates, the eccentric cam 224 rotates eccentrically so that the driving bearing 228 travels along the inner upper surface of the elevating hole 234 to one side (the right side), and the sliding block 230 is elevated, as shown in FIG. 3II. When the eccentric cam 224 is continuously rotated, the driving bearing 228 travels along the inner upper surface of the elevating hole 234, so that the rotating shaft 212 and the eccentric shaft 226 are perpendicular to each other. The driving bearing 228 travels to the other side (the left side) at the upper inner surface of the elevating hole 234. Here, the rotating shaft 212 and the eccentric shaft 226 form an angle of 120°, as shown in FIG. 3III. When the rotating shaft 212 and the eccentric shaft are perpendicular, the driving bearing 228 does not contact the sides of the elevating hole 234.

When the geared motor 210 rotates further, the driving bearing 228 moves to the other side of the elevating hole 234, so that the rotating shaft 212 and the eccentric shaft 226 form 180°. Here, the sliding block 230 is at the highest point, as shown in FIG. 3IV.

When the driving bearing 228 continues its upward and downward movement within the elevating hole 234, the driving bearing moves to one side or the other, so that the sliding block 230 is elevated and lowered to elevate and lower the thermoceramic unit 100. That is, the driving bearing 228 and the sliding block 230 are in contact at all times during the elevating movement, thereby preventing the emitting of noise.

As described above, a sliding block inserted within a square guiding block is elevated by using an eccentric shaft with a sliding movement. Therefore, noise emissions can be blocked for more quieter use of a thermoceramic unit in a thermotherapy apparatus.

Also, a geared motor is used in the guiding block to elevate the sliding block, for a simple configuration that prevents malfunctions and allows mass-production of the device.

It will be apparent to those skilled in the art that various modifications and variations can be made in the present invention. Thus, it is intended that the present invention covers the modifications and variations of this invention provided they come within the scope of the appended claims and their equivalents.

What is claimed is:

1. A lifter (200) of a thermoceramic unit (100) for a thermotherapy apparatus, for lifting and lowering the thermoceramic unit (100) at a predetermined location along a lengthwise movement of the thermoceramic unit (100), the lengthwise movement enabled by a conveyor in the thermotherapy apparatus, the lifter (200) comprising:
a box-shaped guiding block (220) open at a side thereof, connected to the conveyor, and installed below the thermoceramic unit (100);
a geared motor (210) installed opposite to the open side of the guiding block (220);
a sliding block (230) installed in the guiding block (220) within which the sliding block (230) is raised and lowered through a converting driving member that converts a rotating movement of the geared motor (210) to a linear movement; and
a cover plate (240) for covering the open side of the guiding block (220).

2. The lifter (200) of claim 1, wherein the sliding block (230) comprises:
a passage (232) formed therein on surfaces proximal to the guiding block (220) for enabling a smooth elevation thereof; and
an elevating hole (234) formed horizontally along a middle of the sliding block (230) for receiving driving force from the geared motor (210) through the converting driving member, wherein
the converting driving member includes an eccentric cam (224) installed on a rotating shaft (212) of the geared motor (210) and inserted into the guiding block (220), an eccentric shaft (226) fitted in an eccentric hole (225) formed in an eccentric portion of the eccentric cam (224), and a driving bearing (228) inserted over the eccentric shaft (226) and mounted in the elevating hole (234).

3. The lifter of claim 2, further comprising a rotating shaft support bearing (222) installed between the eccentric cam (224) and the guiding block (220), for enabling a smooth rotation of the eccentric cam (224).

4. The lifter of any one of claims 1 through 3, further comprising:
a movement detecting pin (236) protruding from the sliding block (230) toward the cover plate (240);
a pin through-hole (246) elongatedly formed in the cover plate (240), the movement detecting pin (236) moving in a vertical direction in the pin through-hole (246); and
and an upper limit switch (244) and a lower limit switch (242) for detecting a vertical displacement of the movement detecting pin (236).
